(19) United States
(12) Patent Application Publication
(10) Pub. No.: US 2011/0288586 A1
(43) Pub. Date: Nov. 24, 2011

(54) Closed-Loop Automated Chiropractic Adjustment Device

(76) Inventor: Christopher M. Auman, Canonsburg, PA (US)

(21) Appl. No.: 13/110,463

(22) Filed: May 18, 2011

Related U.S. Application Data
(60) Provisional application No. 61/345,693, filed on May 18, 2010.

(51) Int. Cl. A61F 5/00

(52) U.S. Cl. 606/242

(57) ABSTRACT

A chiropractic evaluation, diagnostic and adjusting system includes a surface having including an elongated slot therein, a number of actuators, each including a rod and a position sensor, and a drive for translating the adjustment actuators along the length the slot. A control system determines a curvature of a spine of a patient by controlled extension and retraction of each rod into and out-of contact with the spine of the patient via the slot during translation of the adjustment actuators along the length of the slot. The control system compares the determined curvature of the spine of the patient to a predetermined spine curvature and controls the drive and each of one or more of the actuators to apply a series of corrective stroke pulses to an area of the patient's spine requiring adjustment via the rod of the actuator.

### CROSS REFERENCE TO RELATED APPLICATION

The present invention claims priority from U.S. Provisional Patent Application No. 61/345,693, filed May 18, 2010, which is incorporated herein by reference.

### BACKGROUND OF THE INVENTION

1. Field of the Invention

The present invention relates generally to chiropractic adjustment of musculoskeletal structures, such as the spine, and more specifically to automated mapping and adjustment of musculoskeletal misalignments and abnormalities by way of an automated chiropractic adjustment device that utilizes sensors, actuators and a control system processing unit to map a human spine and make necessary corrective adjustments based on sensor input data.

2. Description of Related Art

The human skeletal system, especially the spine, is a complex and intricate structure. However, like many structures, it is not perfect and virtually everybody experiences skeletal malfunctions at one time or another in their lives. Of particular complexity and concern is the spine, which inherently experiences the most malfunctions of the skeletal system.

It is claimed by many chiropractors that most ailments, from common colds, hearing loss, mental health, and virtually everything in between can be linked to spinal misalignment. These claims have yet to be verified, but given the complexity of the related systems that operate in and around the spine, the claims are not unfounded nor have they been disqualified. While many skilled and talented chiropractors employ their professional trade many times daily with great success, the fact remains that there are few scientific measurement and alignment techniques available to quantify the efforts of the chiropractor.

The Chiropractic art is inherently sensitive to the technique, capability and skill of the individual practitioner. These undefined variables lack technology and scientific data needed for specific, quantifiable analysis and measurement methods and systems to evaluate the success and effectiveness of treatments.

Many of the advancements in chiropractic are simple improvements to dated and existing equipment designs that haven't changed much in nearly half a century. Some of the more advanced improvements assist the practitioner in analyzing the patient, however, other than empirical data, there still lacks quantifiable scientific data to support the methods and techniques applied by chiropractors.

There are ongoing attempts to create an ‘all-encompassing’ chiropractic device, but the majority of attempts have only addressed one particular area of chiropractic treatment, either analysis or adjustment. There are currently devices that have addressed both aspects, however using differing philosophies. A device by Johnson/Axiom Worldwide Inc, for example, uses a closed-loop feedback system to measure tension on a chord related to tension in the spine and correspondingly adjusts distraction forces to the body to compensate. A system by PulStar FRAS system and others like it, measure tissue temperature to determine the troubled areas around the spine and the operator manually transmits subsequent pulse therapy to the identified areas

### SUMMARY OF THE INVENTION

The present invention is a tool for evaluation, diagnosis and correction of chiropractic patient symptoms, problems, and/or injuries.

The present invention is a system that analyzes the precise existing alignment of each vertebrae in the spine, computes the results of the analysis, and follows up with minute mechanical adjustments to the spine and reiterates as necessary. This is accomplished by manually mapping the contours and condition of the human spine through the use of displacement measurement means and, also or alternatively, temperature sensing and/or tissue compliance sensors. A central processing unit collects and analyzes the mapped data, creates visual and graphical displays and develops a treatment protocol. The treatment protocol is delivered to the spine by making necessary adjustments through the use of one or more precision actuators. The system can then repeat itself and make any necessary follow-up adjustments. The system continues to evaluate and correct until it measures a spinal mapping that is within established acceptable parameters.

More particularly, the invention is a chiropractic evaluation, diagnostic and adjusting system comprising: a patient receiving surface supported by a frame, said patient receiving surface including an elongated slot therein; a plurality of actuators, each actuator including an extendable and retractable rod and a position sensor; a drive coupled between the frame and the adjustment actuators, said drive operable for translating the adjustment actuators along the length the slot; and a control system operative for: determining a curvature of a spine of a patient lying on the patient receiving surface by controlled extension and retraction of each rod into and out-of contact with the spine of the patient via the slot while the drive translates the adjustment actuators along the length of the slot; for comparing the determined curvature of the spine of the patient to a predetermined model curvature of the spine to determine an area of the patient's spine requiring adjustment, for controlling the drive to position the actuators adjacent the area of the patient's spine, and for controlling each of one or more of the actuators to apply a series of corrective stroke pulses to the area of the patient's spine via the rod of the actuator.

The feedback from each actuator can enable the control system to determine when the rod of the actuator is in contact with the patient's spine.

The system can further include at least one of the following coupled to the drive and translatable thereby along the length of the slot in contact with or in close proximity to the patient's spine adjacent the patient's spine: a temperature sensor operative for supplying temperature measurements of the patient's spine to the control system while translating along the length of the slot; a pressure sensor operative for supplying pressure measurements of the patient's spine to the control system while translating along the length of the slot; or an electrical impulse sensor operative for supplying electrical measurements of the patient's spine to the control system while translating along the length of the slot.

The invention is also a chiropractic evaluation, diagnostic and adjusting method comprising: (a) under the control of a control system, causing rods of a plurality of actuators to move into and out-of contact with a patient's spine via a slot in a surface upon which the patient is lying posterior to the surface while moving the actuators along the length of the patient's spine; (b) the control system determining a curvature of the patient's spine based on the moving in step (a); (c) the control system determining an area of the patient's spine requiring adjustment based on a comparison the curvature of the patient's spine determined in step (c) to a predetermined spine curvature; and (d) the control system positioning the actuators adjacent the area of the patient's spine and causing the rod of each actuator to apply a series of corrective pulses to the area of the patient's spine.

The method can further include the control system determining a temperature profile of the patient's spine, wherein determining the area of the patient's spine requiring adjustment in step (c) is further based on the determined temperature profile of the patient's spine.

The method can further include the control system determining a pressure profile of the patient's spine, wherein determining the area of the patient's spine requiring adjustment in step (c) is further based on the determined pressure profile of the patient's spine.

The method can further include the control system determining an electrical profile of the patient's spine, wherein determining the area of the patient's spine requiring adjustment in step (c) is further based on the determined electrical profile of the patient's spine.

### BRIEF DESCRIPTION OF THE DRAWINGS

FIG. 1 is a perspective view of a frame of an adjustment table in accordance with the present invention;

FIG. 2 is a top view of an embodiment of the adjustment table, wherein an access slot is visible through which sensors and adjustment devices access the patient;

FIG. 3 is an end view of the adjustment table shown in FIG. 2;

FIG. 4A is a cross-section taken along lines IV-IV in FIG. 2;

FIG. 4B is an illustration of a patient positioned on the cross-section shown in FIG. 4A;

FIG. 5 is a partial perspective view of the frame of the adjustment table in FIG. 1 including an optional ‘Y’ axis adjustment device;

FIG. 6 is a cross-section taken along lines VI-VI in FIG. 4A;

FIG. 7 is a cross-section taken along lines VII-VII in FIG. 6;

FIG. 8 is a block diagram of a control system for the adjustment table of FIG. 1 including a personal computer (PC);

FIG. 9 is a PC based control system;

FIG. 10 is a flow chart of the operation of the adjustment table;

FIG. 11 is a cross-section of one actuator 18 shown in FIG. 6 with internal displacement sensors and body located mounting points;

FIG. 12 is a cross section of an actuator 20 shown in FIG. 6 with internal displacement sensors and rod mounting feature;

FIG. 13 is a cross-section of the horizontal ‘X’ linear drive 4 shown in FIG. 1 with internal displacement sensor;

FIG. 14 is a cross-section of a vertical ‘Z’ linear drive 16 shown in FIG. 1 with internal displacement sensor; and

FIG. 15 is a cross-section of an optional lateral ‘Y’ linear drive 32 with internal displacement sensor.

### DETAILED DESCRIPTION OF THE INVENTION

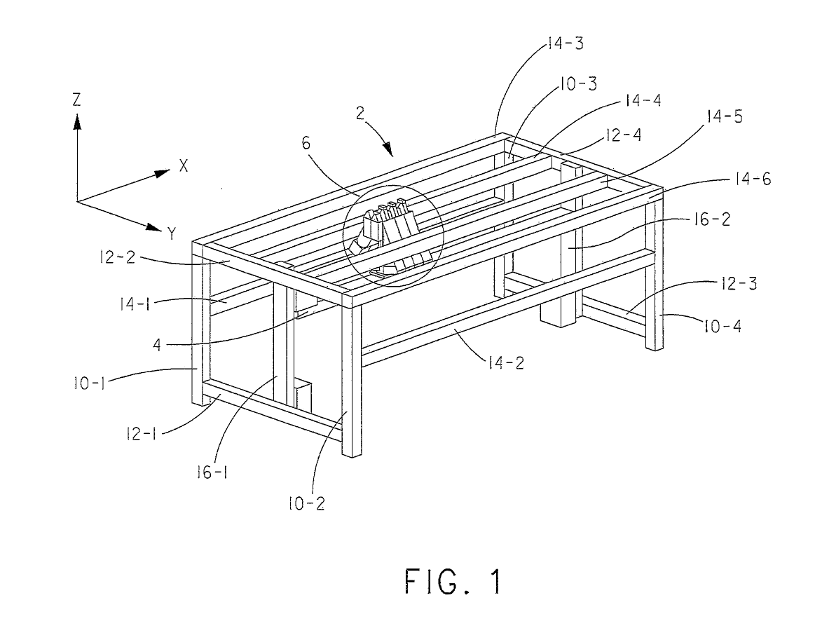

The present invention will be described with reference to the accompanying figures where like reference numbers correspond to like elements. In the figures, reference numbers followed by a dash (“-”) and a number indicate different instances of the same element. For example, the four vertical support legs 10 in FIG. 1 are denoted by 10-1, 10-2, 10-3, and 10-4. The discussion of elements generally by a main reference number in the specification and the illustration of multiple instances of the same element in the figures will be adhered to hereinafter.

With reference to FIGS. 1, 3, and 4, a frame 2 is formed by the assembly of four vertical support legs 10, four short horizontal support members 12 and six long horizontal support members 14.

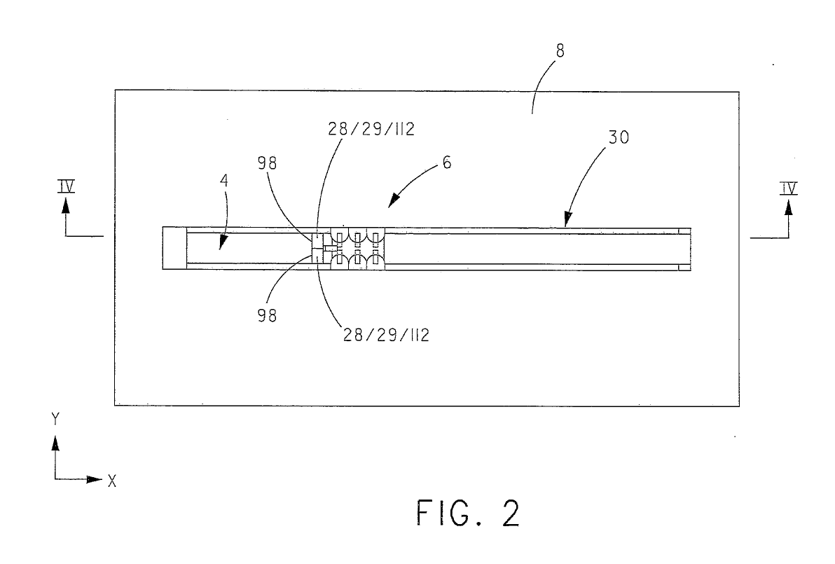

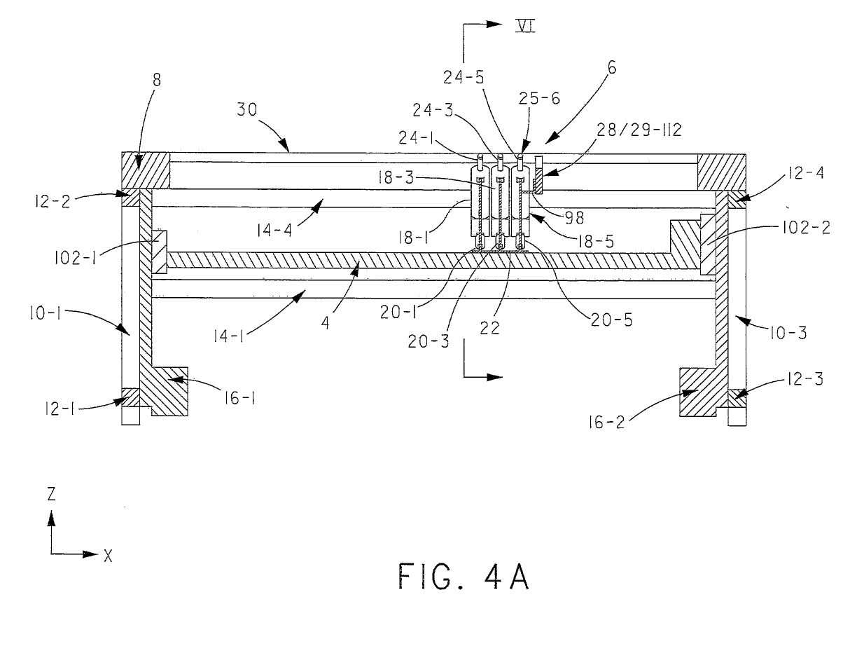

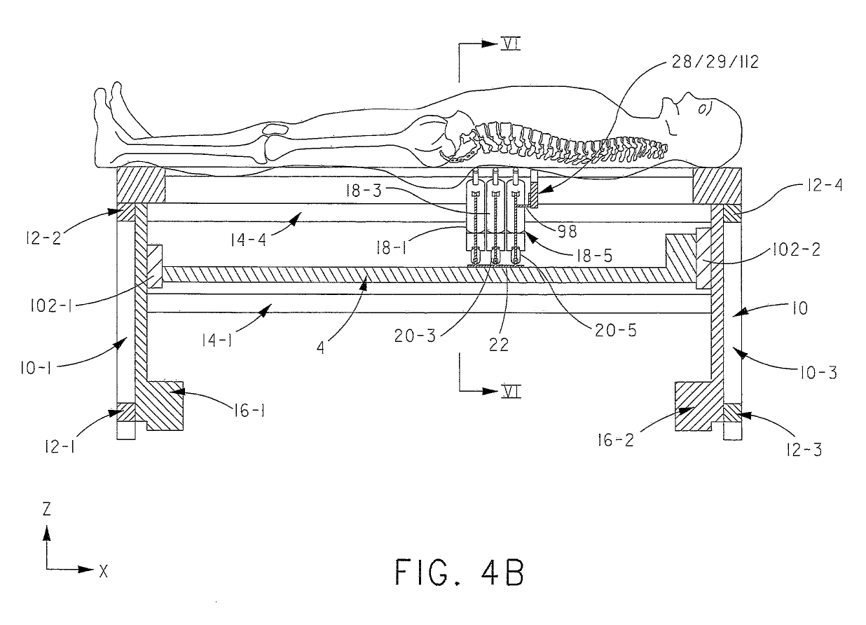

With reference to FIGS. 2-4, a support pad 8 provides comfort to a patient (shown in FIG. 4B) and allows the patient to rest in a manner such as to provide for the patient's spinal column to be in a relaxed state of natural curvature in vertical alignment with an access slot 30 (FIG. 2) along the length of support pad 8 in the ‘X’ direction. The material may be a product such as memory foam. However, pad 8 is not to be construed as limited to memory foam or any other material past or present, known or unknown. Neither shall the structure of the support pad 8 be limited to shape, profile, or otherwise.

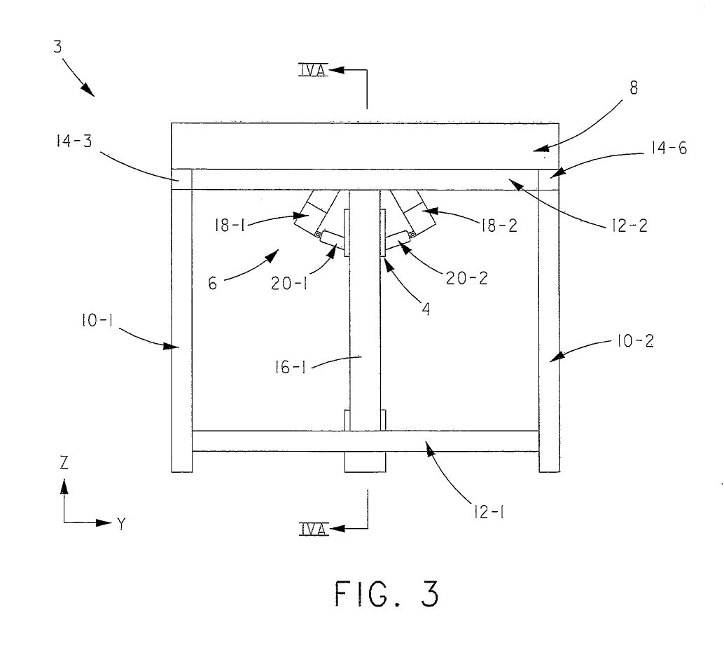

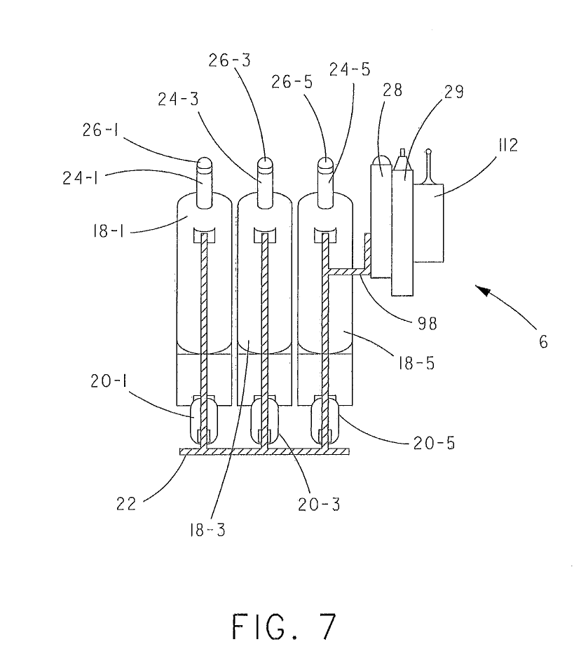

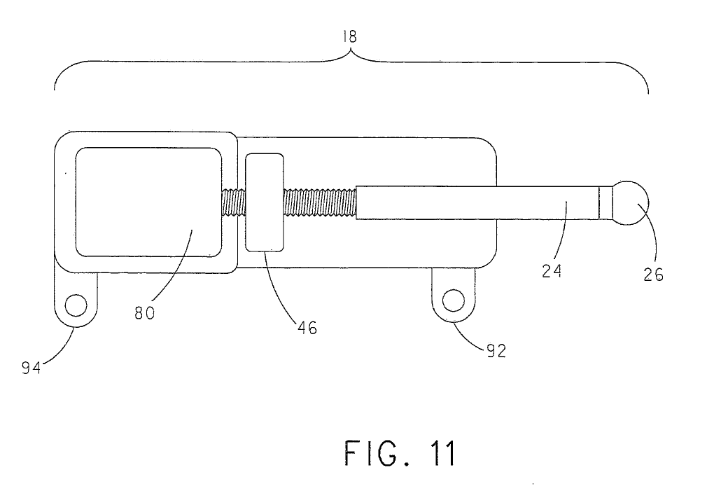

With reference to FIGS. 3-7 and 11, electrical motor 80 driven linear displacement adjustment actuators 18 with internal position sensors 46 are employed as adjustment tools as well as position indicators. One example of a suitable actuator 18 is available from Tolomatic, Inc. and is marketed under the trade name SmartActuator™. However, this is not to be construed as limiting the invention since any suitable and/or desirable actuator with internal motion indexing, controllable thrust, and position sensing that can be utilized as an actuator 18. The adjustment actuator 18 rod 24 will mount a resilient caster style adjustor tip 26 or similar device for contact with the patient's back.

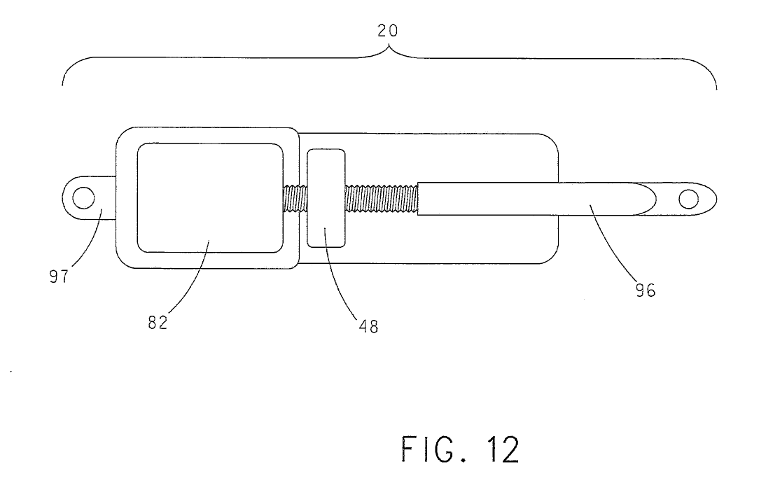

With reference to FIGS. 3-7 and 12, electric motor 82 driven linear actuators with internal position sensors 48 are used as position actuators 20.

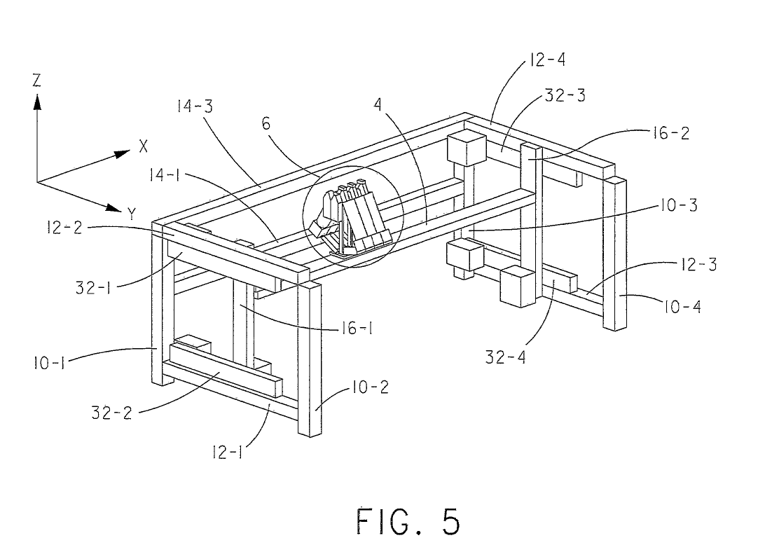

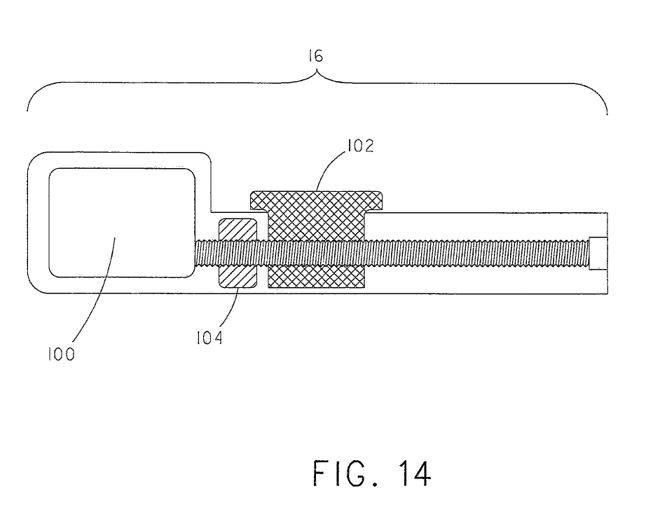

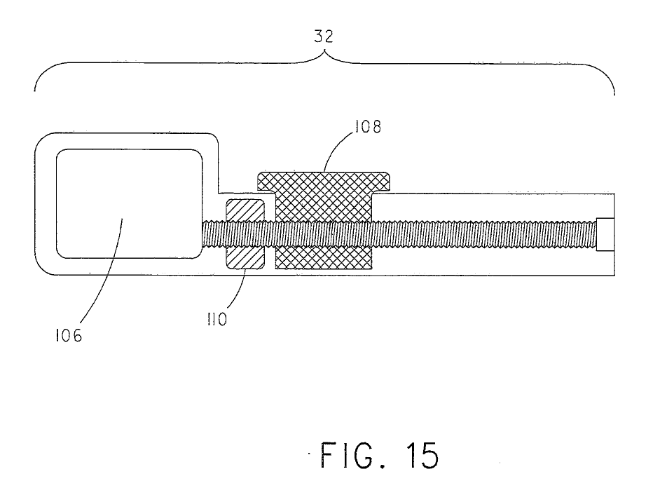

Referring now to FIGS. 1, 3-5 and 13-15, two vertically mounted linear drives 16 control motion in the Z direction and a single horizontally mounted linear drive 4 controls motion in the ‘X’ direction. Internal position sensors 88 and 104 are used to accurately position carrier plates 86 and 102 of drives 4 and 16, respectively. In FIG. 5, an optional ‘Y’ axis control can be integrated through the addition of four horizontal linear drives 32 each of which includes at least one internal position sensor 110 that is utilized to accurately position a carrier plate 108. One example of a suitable linear drive that can be utilized for linear drive 16, 4 and/or 32 is available from Festo Corporation of Hauppauge, N.Y. under the mark DGE. However, this is not to be construed as limiting the invention inasmuch as it is envisioned that any suitable and/or desirable linear drive with internal position sensors, accurate positioning, and programmable control capabilities can be utilized for any one or all of drives 16, 4, and/or 32. As shown in FIG. 15, each horizontal linear drive 32 includes a motor 106, a position sensor 110, and a mounting plate 108. Desirably, the adjustment table includes linear drive 4 for movement of adjustor assembly 6 in the ‘X’ direction, and one or more linear drives 16 for movement of adjustor assembly 6 in the ‘Z’ direction. Adjustment table can desirably, but optionally, include one or more linear drives 32 for optional movement of adjustor assembly 6 in the ‘Y’ direction.

Referring now to FIGS. 4-8, a control system 34 includes a controller 36 to receive input signals from one or all of the sensors, process the received signals, calculate a manipulation protocol 74 and deliver 76 the action signals of the derived protocol. A control system 34 includes controller 36, software, computer 38, and programs. Control system 34 can be located remotely or mounted within the envelope of the entire assembly.

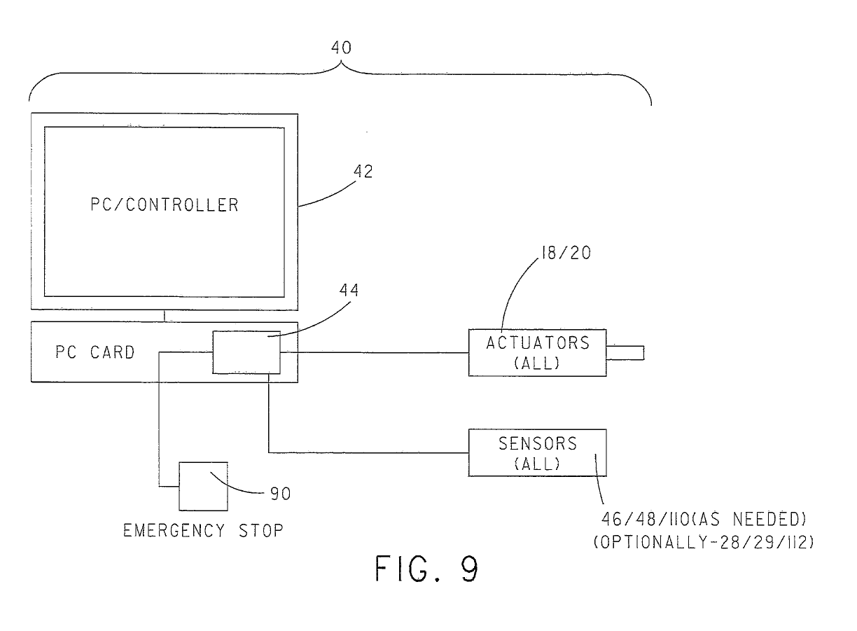

FIG. 9 illustrates an alternate control system 40 that may be utilized wherein PC/Controller 42 includes a PCI card 44 and serves as the controller, monitor, and data collection and processing device in its entirety.

Referring now to FIGS. 4-9, temperature sensors 28, pressure sensors 29 and electrical impulse sensors 112 can optionally be used to identify conditions or parameters in the system. Desirably temperature sensors 28 are used to sense temperature, pressure sensors 29 measure surface tension related to the patient's spine and surrounding tissue, and electrical impulse sensors 112 detect electrical impulse activity in the patient's spine and surrounding tissue.

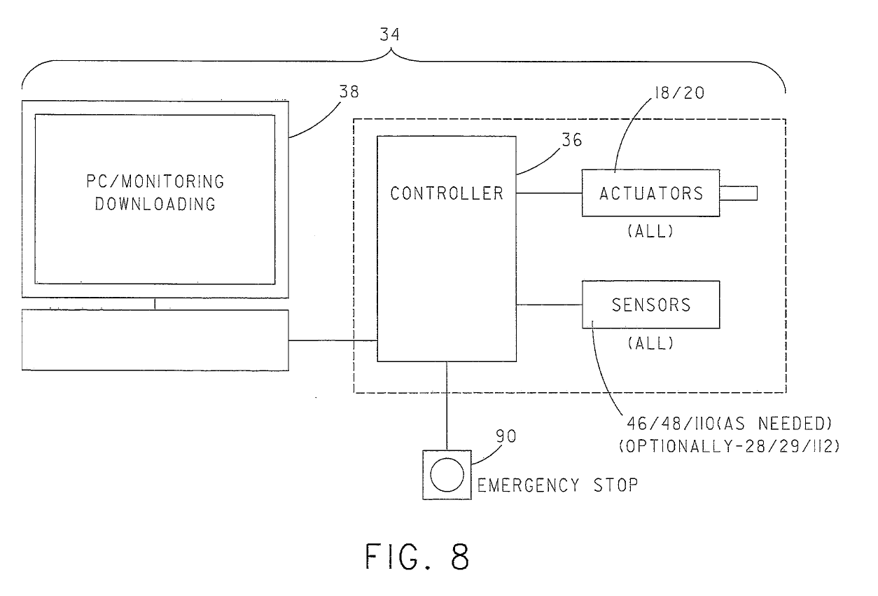

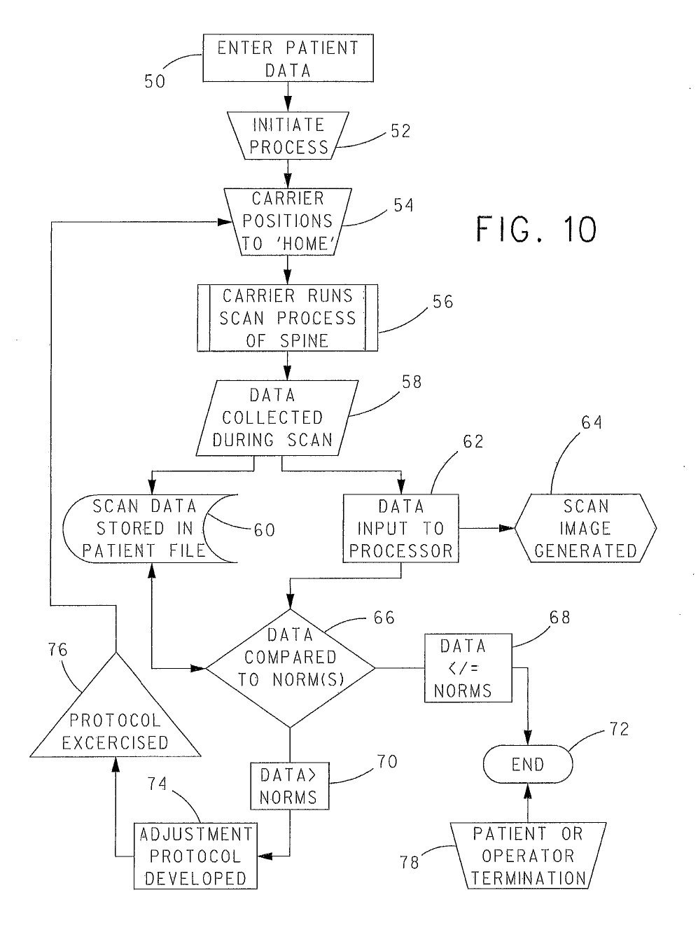

With reference to FIG. 10, the programming logic of control system 34 follows the flow chart sequence shown in FIG. 10. In FIG. 10, patient data 50 is entered into control system 34. This patient data 50 can include personal information, medical history, height, weight, and any information desirable to the practitioner. Initiate Process 52 is initiated by a ‘RUN’ sequence selected from control system 34. Carrier position Home 54 is the beginning or home position of horizontal linear drive 4. Scan Process 56 is where linear drive 4 causes carrier 22 to translate in at least the ‘X’ direction while data is being collected at multiple points along the patient's spine and/or surrounding tissue as discussed hereinafter. It should be appreciated that the ‘X’, ‘Y’, and ‘Z’ directions referred to herein are shown in FIG. 1. Collected Data 58 includes more of the following: measured positions from the position sensors 46 and/or 48, temperature from temp sensors 28, electrical impulses from electrical impulse sensors 112, and tissue compliancy measurements from pressure sensors 29. Data Storage 60 files the scanned information into an individual patient file for future reference. Data Input 62 sends the scanned information to the control system 34 PC 38. Scan image 64 is an image of the patient's spine that can be displayed on a display of PC 38. Data comparison 66 is where the PC 38 compares the scanned data 58 to one or more previously established norms, patient data 50, and/or previous patient scans stored in data storage 60. Data less than or equal to one or more norms 68 is an end process 72 trigger. Data greater than one or more norms 70 is another process trigger. End Process 72 suspends operation. Adjustment Protocol Developed 74 is initiated from process trigger 70, wherein the PC 38 and/or controller 36 develop a specific adjustment procedure for the patient based on the data comparison 66. Protocol Exercised 76 is the process of the adjustment procedure being carried out on the patient. Elected Termination 78 is an emergency stop button 90 (FIG. 8) that can be activated by the patient or practitioner for any reason.

In an embodiment of the present invention, two end assemblies 3 (FIG. 3) are created by two vertical support legs 10 with two short horizontal support members 12 connected between them, one being flush with the top ends of the two vertical support legs 10, the other being flush with the sides and approximately twelve inches from the bottom ends of the two vertical support legs 10. The two end assemblies 3 are now connected with six long horizontal support members 14-1-14-6 (FIG. 1). Two of the long horizontal support members 14-3 and 14-6 are mounted flush with the top ends and outside edges of the vertical support legs 10, while two more 14-4 and 14-5 are mounted flush with the top edges of the upper short horizontal support members 12 and each spaced approximately twelve inches from the outside mounted long horizontal support members 14-3 and 14-6. Two more long horizontal support members 14-1 and 14-2 are mounted between vertical support legs (10-1 and 10-3) and (10-2 and 10-4), being flush with the outside edges and approximately twelve inches from the bottom ends of the vertical support legs 10-1-10-4. Support pad 8 is mounted atop of the four upper long horizontal support members 14-3-14-6.

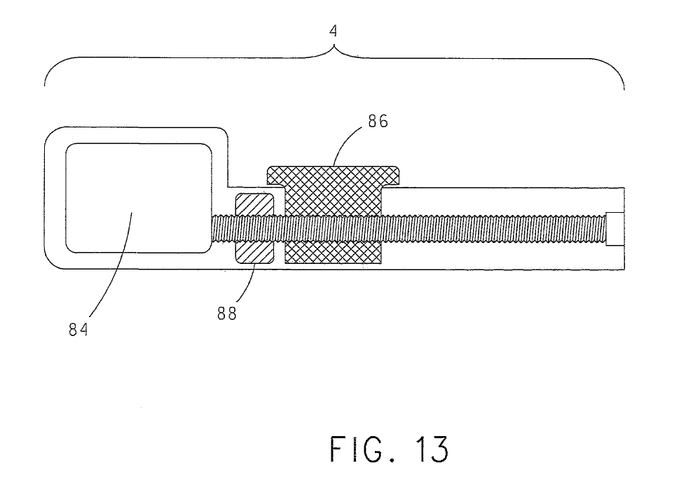

One vertical linear ‘Z’ drive 16 mounts to the inside of each end assembly 3 by securing it to the corresponding two short horizontal support members 12. The long horizontal linear ‘X’ drive 4 is mounted by its ends to plate 102-1 and plate 102-2 (FIGS. 4A and 4B) of the vertical linear ‘Z’ drives 16-1 and 16-2, respectively. Carrier frame 22 mounts to plate 86 (FIGS. 4A and 13) of horizontal linear ‘X’ drive 4.

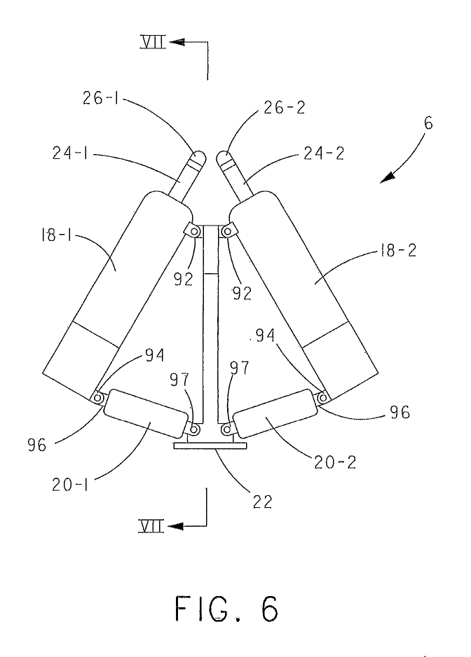

Adjustor tips 26 mount to the distal ends of adjustor rods 24 of adjustment actuators 18. Adjustment actuators 18 are mounted to carrier frame 22 at their upper pivot points 92 (FIG. 6) and mounted to position actuators 20 thrust rods 96 at their lower pivot points 94. The other ends 97 of position actuators 20 are mounted to carrier frame 22. One temperature sensor 28, one pressure sensor 29, and one electrical impulse sensor 112 are mounted to the carrier frame 22 mounting bracket 98. As shown in FIG. 2 a second temperature sensor 28, a second pressure sensor 29, and a second electrical impulse sensor 112 can be mounted to the same mounting bracket 98 or a different mounting bracket 98 whereupon temperature sensors 28, pressure sensors 29, and electrical impulse sensors 112 are positioned on either side of the center line of slot 30. However, this is not to be construed as limiting the invention. For the purpose of description hereinafter, it will be assumed that inventive adjustment table includes only one temperature sensor 28, one pressure sensor 29, and one electrical impulse sensor 112.

The operational flow process of the above-described system as outlined in FIG. 10 begins with the entry of patient data 50 into PC 38. Next, as shown in FIG. 4B, the patient lies down on support pad 8 with the patient's spine centered over the access slot 30 and posterior to the table. Desirably, the patient relaxes to allow the support pad 8 to conform to the natural curvature of the patient's spine. At a suitable time, the practitioner will initiate RUN process 52. Originating from ‘home’ 54 position, adjustor assembly 6 begins an initial scan process 56 selected by controller 36 or the practitioner. The scan begins by locating the position of the patient's skull by extending the rods 24 of adjustment actuators 18 until adjustor tips 26 make contact with the patient's skull and controller 36 records the positions of tips 26 via internal position sensors 46. The location where each tip 26 contacts the patient's skull can be adjusted by controlling the extension or retraction of the rod 24 of the position actuator 20 that is coupled to the adjustment actuator 18 that supports the rod 24 that in-turn supports the adjuster tip 26. Via controlled motion of linear drive 4 adjustor assembly 6 slowly translates the length of the patient's spine. During translation of adjustor assembly 6 along the length of the patient's spine, position and optionally temperature, and tissue compliance readings are taken on and/or around the spine via position sensors 46 of adjustment actuators 18, position sensors 48 of position actuators 20, internal position sensor 104 of linear ‘Z’ drives 16, position sensors 88 of linear ‘X’ drive 4 temperature sensors 28, and pressure sensors 29. If desired, carrier frame 22 can support a sensor bracket 98 on either side of the patient's spine, with each sensor bracket supporting a temperature sensor 28, a pressure sensor 29, and/or an electrical impulse sensor 112. However, this is not to be construed as limiting the invention since it is envisioned that a single sensor bracket supporting one temperature sensor 28, one pressure sensor 29 and/or one electrical impulse sensor 112 at any suitable and/or desirable location on or about the patient's spine can be utilized.

During translation, displacement sensors 46 and 48 map the contours and location of each spinal element as optional temperature sensor 28, optional pressure sensor 29 and/or optional electrical impulse sensor 112 identify areas of probable injury or resistance. Each temperature sensor 28, pressure sensor 29, and/or electrical impulse sensor 112 reading will correlate to the corresponding coordinates of the adjustment actuator 18, vertical linear ‘Z’ drive 16 and horizontal linear ‘X’ drive 4 at a specific location for position indexing along the spine. To this end, because sensor bracket 98 is rigidly originally affixed to carrier frame 22, the spatial position of each actuator tip 26 relative to temperature sensor 28, pressure sensor 29, and/or electrical impulse sensor 112 is known and can be taken into account when mapping the temperature profile, pressure profile and/or electrical impulse profile of the patient's spine via the readings taken during translation of temperature sensor 28, pressure sensor 29, and/or electrical impulse sensor 112 along the length of the patient's spine. Sensor data 58 is then analyzed by PC 38 and controller 36 processors to create one or more initial models from the data 58. These initial models can include: a temperature profile of the patient's spine, a pressure profile of the patient's spine, an electrical impulse profile of the patient's spine, a two or three dimensional spatial profile of the patient's spine, or some combination thereof. In practice, it is envisioned that the spatial profile of the patient's spine determined from data 58 acquired from one or more of the position sensors 46 and/or 48 during translation of adjustor assembly 6 along the patient's spine will form the primary basis of adjustment of the patient's spine to be discussed hereinafter. However, this is not to be construed as limiting the invention since it is envisioned that data acquired from temperature sensor 28, pressure sensor 29, and/or electrical impulse sensor 112 can also be utilized in the formulation of an adjustment protocol. In this regard, it is to be appreciated that the use of temperature sensor 28, pressure sensor 29, and/or electrical impulse sensor 112 in the inventive adjustment table is/are optional.

Control system 34 software then evaluates model data 58 against standard norms or spatial/position profiles 66 for spinal curvature, length, and other various parameters based on specific patient data input, such as height, weight, sex, etc. The software compares the model data 58 against a theoretical model, as well as calculates the angle of orientation from one vertebra to the next from the measured data 58 to determine trouble areas of the patient's spine and to develop a corresponding course of corrective protocol 74. Once the analysis 66 is complete, the control system dispatches the corrective action signals 76 to the adjustor assembly 6, linear drives 4, 16, and/or 32, and motors 80 and 82 of actuators 18 and 20, respectively.

Under the control of control system 34, plate 86 of linear drive 4 locates carrier frame 22 and adjustor actuators 18 to the determined trouble areas of the patient's spine and position actuators 20 adjust for angles of correction. At a suitable time, control system 34 causes one or more adjustor actuator 18 rods 24 to extended to locate the contact point on the spine and one or more adjustment actuators 18 is/are moved into final position(s). Once each desired adjustment actuator 18 is in its final position, control system 34 causes the adjustment actuator 18 to rapidly move the corresponding adjustor tip 26 rapidly into and out of contact with the patient's spine in a series of rapid fire corrective stroke pulses. The stroke pulses are controlled in amplitude, force, duration, and quantity by the calculated corrective protocol 74. The process of applying a series of rapid fire corrective stroke pulses to the patient's spine is repeated as necessary for each determined trouble area of the patient's spine by translation of adjustor assembly 6 along the length of the patient's spine, with adjuster assembly 6 stopping at each determined trouble area to allow the series of rapid fire corrective stroke pulses to be applied to the patient's spine. Once the adjustor assembly 6 has translated the length of the spine making all of the required adjustments, it resets 54 to the home position and performs another analysis 56. This new data 58 is now compared to the theoretical model and/or to one more previous scan data 60. If a comparison of this new data to the theoretical model and/or to one or more previous scans indicates that further adjustment of the patient's spine is needed, control system 34 causes one or more adjustment actuators 18 to apply another series of corrective stroke pulses to the new trouble areas determined by the comparison of the new data to the theoretical model and/or one or more previous scanned data 60. This iterative process increases the accuracy of the system on each pass and continues until the measured data and the theoretical data reach equilibrium 68 or the program is terminated 78 by the operator or the patient.

During evaluation, the actuator's position sensors 46, 48 determine the location of each vertebra in three planes and the processor 38 of control system 34 evaluates the position of each vertebra in relation to the others. Once all vertebrae have been mapped, processor 38 evaluates the curves of the mapped spine data 58 against predetermined curvature limits. The optional temperature sensor 28, pressure sensor 29, and/or electrical impulse sensor 112 serve as verification and backup analysis of the mapped vertebral elements.

This invention has been described with reference to exemplary embodiments. Obvious modifications and alterations will occur to others upon reading and understanding the preceding detailed description. It is intended that the invention be construed as including all such modifications and alterations insofar as they come within the scope of the appended claims or the equivalents thereof.

The invention claimed is:

1. A chiropractic evaluation, diagnostic and adjusting system comprising:
a patient receiving surface supported by a frame, said patient receiving surface including an elongated slot therein;
a plurality of actuators, each actuator including an extendable and retractable rod and a position sensor;
a drive coupled between the frame and the adjustment actuators, said drive operable for translating the adjustment actuators along the length the slot; and
a control system operative for:
determining a curvature of a spine of a patient lying on the patient receiving surface by controlled extension and retraction of each rod into and out-of contact with the spine of the patient via the slot while the drive translates the adjustment actuators along the length of the slot;
for comparing the determined curvature of the spine of the patient to a predetermined model curvature of the spine to determine an area of the patient's spine requiring adjustment,
for controlling the drive to position the actuators adjacent the area of the patient's spine, and
for controlling each of one or more of the actuators to apply a series of corrective stroke pulses to the area of the patient's spine via the rod of the actuator.

2. The system of claim 1, wherein feedback from each actuator enables the control system to determine when the rod of the actuator is in contact with the patient's spine.

3. The system of claim 1, further including at least one of the following coupled to the drive and translatable thereby along the length of the slot in contact with or in close proximity to the patient's spine adjacent the patient's spine:
a temperature sensor operative for supplying temperature measurements of the patient's spine to the control system while translating along the length of the slot;
a pressure sensor operative for supplying pressure measurements of the patient's spine to the control system while translating along the length of the slot; or
an electrical impulse sensor operative for supplying electrical measurements of the patient's spine to the control system while translating along the length of the slot.

4. A chiropractic evaluation, diagnostic and adjusting method comprising:
(a) under the control of a control system, causing rods of a plurality of actuators to move into and out-of contact with a patient's spine via a slot in a surface upon which the patient is lying posterior to the surface while moving the actuators along the length of the patient's spine;
(b) the control system determining a curvature of the patient's spine based on the moving in step (a);
(c) the control system determining an area of the patient's spine requiring adjustment based on a comparison the curvature of the patient's spine determined in step (c) to a predetermined spine curvature; and
(d) the control system positioning the actuators adjacent the area of the patient's spine and causing the rod of each actuator to apply a series of corrective pulses to the area of the patient's spine.

5. The method of claim 4, further including the control system determining a temperature profile of the patient's spine, wherein determining the area of the patient's spine requiring adjustment in step (c) is further based on the determined temperature profile of the patient's spine.

6. The method of claim 5, further including the control system determining a pressure profile of the patient's spine, wherein determining the area of the patient's spine requiring adjustment in step (c) is further based on the determined pressure profile of the patient's spine.

7. The method of claim 6, further including the control system determining an electrical profile of the patient's spine, wherein determining the area of the patient's spine requiring adjustment in step (c) is further based on the determined electrical profile of the patient's spine.

8. The method of claim 4, further including the control system determining a pressure profile of the patient's spine, wherein determining the area of the patient's spine requiring adjustment in step (c) is further based on the determined pressure profile of the patient's spine.

9. The method of claim 8, further including the control system determining an electrical profile of the patient's spine, wherein determining the area of the patient's spine requiring adjustment in step (c) is further based on the determined electrical profile of the patient's spine.

10. The method of claim 4, further including the control system determining an electrical profile of the patient's spine, wherein determining the area of the patient's spine requiring adjustment in step (c) is further based on the determined electrical profile of the patient's spine.
